(19) United States
(12) Patent Application Publication
(10) Pub. No.: US 2005/0059731 A1
(43) Pub. Date: Mar. 17, 2005

(54) Erythrosin-based antimicrobial photodynamic therapy compound and its use

(75) Inventors: Volker Albrecht, Jena (DE); Burkhard Gitter, Jena (DE)

Correspondence Address: BOLESH J. SKUTNIK PhD.JD, 515 Shaker Road, East Longmeadow, MA, 01028, US

(73) Assignee: CeramOptec Industries, Inc.

(21) Appl. No.: 10/860,297

(22) Filed: Jun. 3, 2004

Related U.S. Application Data
(60) Provisional application No. 60/503,339, filed on Sep. 16, 2003.

(51) Int. Cl. A61K 31/366; A61N 1/30

(52) U.S. Cl. 514/453; 604/20

(57) ABSTRACT

A method and composition for destroying microbes, especially bacteria, in the body utilizing Erythrosin B in conjunction with electromagnetic radiation is disclosed. In a preferred method, a composition comprising Erythrosin B is introduced to a treatment area. After a sufficient period of time has elapsed, radiation of a suitable wavelength is applied to the area to activate the Erythrosin B and by a photodynamic reaction to destroy the bacteria. Preferred radiation has a wavelength around 530 nm. Erythrosin B is incorporated within a gel, which acts to restrict the photodynamic action proximate to the biofilm, thus ensuring that only unwanted bacteria is effected and natural microflora is unharmed. This method is effective for destroying at least Gram-positive bacteria, and is particularly effective in areas where complex media such as saliva are also present.

### DOMESTIC PRIORITY UNDER 35 USC 119(e)

This application claims the benefit of U.S. Provisional Application Ser. No. 60/503,339, filed Sep. 16, 2003, which is incorporated by reference herein.

### BACKGROUND OF THE INVENTION

1. Field of the invention

The invention relates to the field of photodynamic therapy, particularly the use of photodynamic therapy in the selective destruction of bacteria in humans and animals.

2. Information Disclosure Statement

Photodynamic therapy (PDT) is well known and has been utilized to combat numerous diseases generally associated with hyperproliferating tissue, such as cancer and various skin conditions. PDT has also been utilized as an antimicrobial treatment. However, there are a number of major problems associated with antimicrobial PDT. The first problem stems from the difficulty of finding photoactive substances that can be effectively used against both Gram-positive and Gram-negative bacteria. Gram-negative bacteria present a much tougher obstacle primarily due to their double-layer outer membrane structure.

The primary difference between Gram-positive and Gram-negative bacteria lies in the cell walls, as is illustrated in FIGS. 1 and 2. As shown in FIG. 1, Gram-positive cells have a thick peptidoglycan cell wall 101, consisting of many individual peptidoglycan layers 103 (for example, 20-40 layers) surrounding cell membrane 105. In contrast, as shown in FIG. 2, Gram-negative cells have only a thin layer of peptidoglycan 201 surrounding cell membrane 203, which is further surrounded by an additional outer membrane 205. This additional layer allows Gram-negative and Gram-positive bacteria to be differentiated using Gram's method. Because of the outer membrane in Gram-negative bacteria, the crystal violet-iodine stain cannot reach the peptidoglycan layer of the cell wall and be retained in Gram-negative bacteria after Gram's method as it is in Gram-positive bacteria. The outer membrane is primarily responsible for inhibiting penetration of many substances into Gram-negative bacteria, and is the reason for the difficulty in finding photosensitizers that are effective against both types of bacteria.

Another problem results from difficulty in finding a suitable photosensitive compound that retains at least some activity in the presence of complex media such as blood serum, blood or saliva. Most photosensitive compounds (photosensitizers) that display good activity against cell suspensions in poor media such as phosphate buffered saline show virtually no effect in the presence of blood serum, blood or saliva. This is the case because the components in these complex media (e.g. proteins, blood cells) compete with the bacteria for affinity of the PDT compound. Yet another problem involves the risk of destroying microorganisms that are naturally occurring and beneficial or necessary to certain bodily functions. Application of anti-microbial PDT runs the risk of destroying the beneficial microflora along with the harmful bacteria that is sought to be eliminated.

Erythrosin B is a red dye that absorbs in the 450-600 nm blue-green range. It is used as a biological stain in processes such as photomicrography. For example, Erythrosin B is used widely as a counterstain to different nuclear stains, in both plant and animal tissue, or as a contrast stain for bacterial cells.

Erythrosin and Erythrosin B can be used as a dye in conjunction with dental treatments to visually indicate the presence and location of plaque on teeth. Erythrosine has been utilized to remove bacteria from biological surfaces, and used in anti-bacterial treatments.

U.S. Pat. No. 4,581,227 also discloses the use of Erythrosine or other substances to remove micro-organisms attached to biological surfaces, for example the stomach and intestines, teeth and surfaces of wounds, of pigs, livestock and poultry. This method does not serve to destroy bacteria, but rather to remove from or prevent adherence of bacteria to biological surfaces.

Erythrosine and related dyes have been used in periodontal treatments which detect and treat microbes and cavities on and around the teeth and gums. U.S. Pat. No. 6,337,357 discloses an antimicrobial caries-detecting composition comprising water, a water-miscible solvent or a combination, a dye capable of staining the caries-infected portions of teeth, and an antimicrobial agent. This is both a cavity detection and sterilization system. A dye such as erythrosine, which is among the suitable dyes that are soluble in the solvent or solvents and capable of visually indicating the presence and location of cavities, may be used. For this invention, Erythrosine is utilized purely as a staining agent and is not contemplated as an anti-microbial agent.

Erythrosin B is a known photosensitizer, utilized in both medical and non-medical treatments. Non-medical treatments include insecticidal treatments and industrial surface treatments, and medical treatments include antimicrobial PDT treatments of teeth and other biological surfaces, and PDT of cancerous and other diseased tissue.

U.S. application No. 2002/0173832 A1 describes a PDT treatment for neovascularization in the eye as a result of age-related macular degeneration. Erythrosin and Erythrosin B are listed among many possible photosensitizers for use in this method.

U.S. Pat. No. 6,609,014 discloses the use of PDT to inhibit restenosis in blood vessels caused by intimal hyperplasia. Among the many photosensitizers purported to be useful in this treatment are Erythrosin and Erythrosin B.

U.S. patent application No. 2002/0022032 A1 discloses a method of using photosensitizers in combination with immuno-adjuvants to destroy metastatic tumor cells. Photosensitizers purported to be of use in the method include xanthene dyes such as Erythrosin and Erythrosin B.

U.S. Pat. No. 4,647,578 discloses water soluble, insecticidal compositions of certain xanthene dye free acids such as Erythrosin B, to combat both adult insects and insect larvae. The insects or larvae are caused to ingest compounds containing these compositions, which cause the insects or larvae to die upon exposure to visible light.

U.S. Pat. No. 5,798,112 describes the use of photoactive dyes such as Erythrosin B in a phototoxic insecticidal composition. The composition contains selected photoactive dyes, a bait, and an adjuvant. The compound is ingested by desired insects, whereby the adjuvant interacts with the photoactive dye and the insect membranes to alter the toxicity of the composition, which acts to kill the insects after exposure to sunlight for a period of time. U.S. Pat. No. 6,506,791 discloses a method of treating protozoan infections in fish. A photoactive dye including Erythrosine B is introduced into an aqueous environment containing infected fish, such that the concentration of the photoactive dyes is sufficient to kill some or all of the bacteria.

European Patent No. 652709 B1 discloses a method of killing bacteria on biofilms by applying certain photosensitizers, including Erythrosin B, to the surfaces and photodynamically inactivating the bacteria. This method is prescribed for use on hard domestic and industrial surfaces such as glass, plastics and ceramics. It does not disclose a use for biological surfaces.

A method of photothermal destruction of oral bacteria is disclosed in U.S. Pat. No. 6,290,496. A formulation containing a dye, preferably Erythrosin B, is applied to the teeth to selective stain oral bacteria. Radiation, filtered so that wavelengths highly absorbed by hemoglobin are excluded, is applied to selectively increase the temperature of the stained bacteria and destroy the bacteria by coagulation. This method does not disclose a way to selectively destroy only harmful bacteria while leaving natural microflora unharmed.

Photosensitizers and PDT methods utilizing halogenated xanthene or their derivatives are described in U.S. patent application No. 2001/0022970 A1, for treatment of conditions in various body tissues including the skin and circulatory systems. Diseases such as cancer and microbial infections can purportedly be treated with the disclosed compositions and methods. Compounds such as rose bengal and Erythrosin B are disclosed as potential photosensitizers. The method involves intracorporeal administration, such as intravenous injection and transcutaneous delivery. The photosensitizer can be incorporated in a gel (par. 46) The invention is applicable to diseases of mouth, application can be directly or indirectly to, or substantially proximate to, tissues including the mouth and gums, for treatment of diseases such as Gum and other periodontal diseases including gingivitis. (par. 69) The medicament can be applied to microbial infections of humans and animals and delivered to or substantially proximate to infected tissues. (par. 97) Exemplary bacteria include streptococci. (par. 98) This invention generally describes the use of photosensitizers such as Erythrosin B in PDT treatments, and does describe their use in oral and anti-bacterial treatments, but does not describe a method or composition that would allow photosensitizers to be restricted to a given area or proximate to a biofilm, such as a gel formulation for direct application to the teeth, gums and/or tongue. Furthermore, this invention does not disclose a method or composition for selectively destroying harmful bacteria while leaving natural microflora unharmed. Lastly, this invention does not disclose a method or composition that ameliorates the deleterious effects of complex media such as blood, blood serum and saliva.

The above PDT methods and/or compositions are disadvantageous in that they can indiscriminately destroy normal microflora present in body areas such as in the mouth. These microflora perform essential functions, and thus any anti-bacterial method/composition should avoid destroying such natural microflora. The state of the art does not address nor solve this problem.

There is a need for an antimicrobial PDT method and compound which is effective in the presence of complex media such as saliva. This method should be effectively used against both Gram-positive and Gram-negative bacteria, but for special applications fields the effective killing of the Gram-positive bacteria is sufficient. Also, the method and compound should be effective against harmful bacteria while leaving necessary bacteria unharmed. The present invention addresses this need.

### OBJECTIVES AND BRIEF SUMMARY OF THE INVENTION

It is an object of the present invention to provide a method for the efficient and selective destruction of harmful microbes, especially bacteria, in human and animal subjects.

It is another object of the present invention to provide an anti-bacterial method that can be controllably and selectively activated by electromagnetic radiation.

It is yet another object of the present invention to provide a method that is effective for the destruction of Gram-positive bacteria.

It is a further object of the present invention to provide an anti-bacterial method and composition that is effective in the presence of complex media such as saliva.

Briefly stated, the present invention provides a method for destroying microbes, especially bacteria, in the body utilizing a composition containing Erythrosin B in conjunction with electromagnetic radiation. In a preferred method, a composition comprising Erythrosin B is introduced to a treatment area. After a sufficient period of time has elapsed, radiation of a suitable wavelength is applied to the area to activate the Erythrosin B and by a photodynamic reaction to destroy the bacteria. Preferred radiation has a wavelength around 530 nm. Erythrosin B is incorporated within a gel, which acts to restrict the photodynamic action proximate to the biofilm, thus ensuring that only unwanted bacteria is affected and natural microflora is unharmed. This method is effective for destroying at least Gram-positive bacteria, and is particularly effective in areas where complex media such as saliva are also present.

The above, and other objects, features and advantages of the present invention will become apparent from the following description read in conjunction with the accompanying drawings.

### BRIEF DESCRIPTION OF FIGURES

FIG. 1—Cross-sectional view of the cell envelope of a Gram-positive bacteria cell.

FIG. 2—Cross-sectional view of the cell envelope of a Gram-negative bacteria cell.

FIG. 3—Graph showing photodynamic inactivation of Streptococcus mutans DSM6178 by Erythrosin B containing gel

FIG. 4—Graph showing survival of Streptococcus spec. after photodynamic inactivation by Erythrosin B containing gel

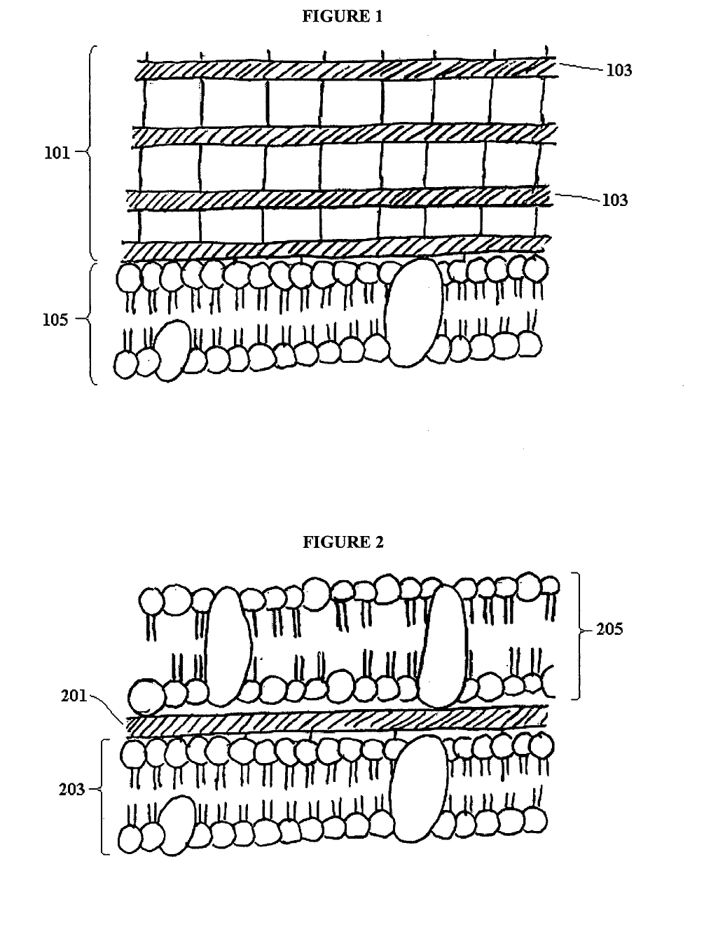

### DETAILED DESCRIPTION OF PREFERRED EMBODIMENTS

Because of the difficulties found in prior art methods and compounds, particularly in avoiding the deleterious effects of complex media such as blood serum, blood or saliva, and in avoiding the destruction of naturally-occuring microflora, it is desirable to find a compound that overcomes these disadvantages. Erythrosin B was found to be an effective photosensitizing substance against Gram-positive bacteria in saliva. This result is very interesting for special application fields, e.g. for effective killing of Streptococcus spec. cells in the oral cavity preventing oral caries. Another advantage noted was that the presence of complex components of the medium (e.g. saliva) do not neutralize the effectiveness of Erythrosin B in targeting bacteria, as is often the case with other photosensitizers. Erythrosin B is thus part of an effective anti-bacterial treatment according to the present invention. An anti-bacterial PDT composition including Erythrosin B is also part of the present invention. In a preferred embodiment, the antibacterial treatment contains three general steps. The first step is to introduce the Erythrosin B composition to an environment containing the bacteria. The second step is to allow a sufficient period of time to elapse to allow the Erythrosin B to penetrate into the bacteria cells in the treatment area or at least bind onto components of their cell envelope. The final step is to apply radiation of a suitable wavelength to initiate a photodynamic mechanism by activation of Erythrosin B, causing the production of reactive oxygen species and free radicals leading to the destruction of the bacteria.

The preferred “exposure time”, or period of time between application of the Erythrosin B composition and irradiation that is sufficient to allow the photosensitizer to diffuse into a biofilm or onto a surface, is variable, and will change depending on factors such as the type of bacteria to be treated, the body area to be treated, and the method of introducing the Erythrosin B composition, respectively. Usually for topical applications, this period will be at least 5 minutes. For treating internal bacterial infections, the composition may be injected into the bloodstream for systemic application, or locally injected if the infection is confined to a specific area. For infections on or near the skin, the composition may be in the form of solution, cream, gel or lotion for topical application.

In a preferred embodiment, the composition of the present invention comprises Erythrosin B contained within a gel. Application of an Erythrosin B gel is advantageous in that the composition can be selectively applied and adhered to surfaces where plaque is present, so that only bacteria located in a biofilm or caries is affected by subsequent irradiation. This is significant in that there are many microorganisms present in the body and on body surfaces that are important to biological processes. It is important that an anti-bacterial treatment avoid killing these natural, beneficial microflora. In the composition of the present invention, the Erythrosin B is restricted to and concentrated to an area near the gel. After the gel is applied to the biofilms, the Erythrosin B diffuses from the gel matrix into the plaque, directly staining the targeted bacteria. Only the bacteria in the plaque are sufficiently stained (the concentration of Erythrosin B is sufficiently high) for application of illumination to stimulate a significant photodynamic effect. Thus, a significant amount of Erythrosin B cannot travel to areas away from the area of application on the biofilm. The area of activation is therefore restricted only to those areas proximate to the biofilm, and thus proximate to the harmful bacteria.

An exemplary treatment according to the present invention is the prophylactic application of the Erythrosin B gel to teeth and/or the dorsum of the tongue to destroy harmful bacteria so that caries do not develop. Alternatively, the gel can be applied to existing caries or diseased tissue to destroy bacteria thereon. The gel is applied to the teeth or other surfaces, such as the gums, and activated by suitable radiation to destroy nearby bacteria in the biofilm. In a preferred embodiment, the biofilms targeted by the present invention are primarily those biofilms located on the teeth and/or the dorsum of the tongue, where harmful bacteria reside that lead to dental caries. Because significant concentrations of Erythrosin B are not present away from the gel composition, other microflora in the mouth are not affected.

There are numerous materials that can be used in the present invention to create a gel formulation. All materials must be non-toxic and approved for internal or oral use. The gel components should solubilize Erythrosin B. Numerous cellulose based gels are contemplated, such as hydroxyethyl cellulose. An exemplary embodiment of a gel of the present invention comprises Erythrosin B, hydroxyethyl cellulose, propyleneglycol, water, and an optional fragrance or aromatic compound.

After a preselected period of time, radiation is applied to the treatment site to activate the Erythrosin B and destroy bacteria. The preferred wavelength of the activating radiation is between 500 nm and 580 nm, and is even more preferably around 530 nm. The radiation can be non-coherent radiation such as from a lamp, or coherent laser radiation. For surface or subsurface treatments, a lamp may be effective in irradiating specific infected areas, whereas for infected areas deeper within the body, an optical fiber apparatus including one or more optical fibers, which may further contain diffusers or other devices as needed to irradiate a certain internal area, is preferred to deliver laser radiation to those internal areas. A preferred laser source is a diode pumped 532 nm laser.

The present invention is further illustrated by the following examples, but is not limited thereby.

### EXAMPLE 1

Photodynamic Inactivation of Bacterial Cell Suspensions of Streptococcus mutans by Erythrosin B:

The organism used in this study was Streptococcus mutans DSM6178 (ATCC 35668). Gram-positive Streptococcus spec. are jointly responsible for the development of oral caries.

Streptococcus mutans cells were grown aerobically overnight at 37° C. in Tryptic Soy Broth (Merck KGaA Darmstadt, Germany). Cells were harvested by centrifugation and resuspended in sterile phosphate-buffered saline (PBS) supplemented with 10% sterile filtered natural saliva. The final OD (Optical Density) at 600 nm, for a 1 cm path length, in all cases was 0.05. About 0.5 ml of an Erythrosin B gel of hydroxyethyl cellulose (1 mM, 2 mM, 3 mM and 8 mM Erythrosin B) were placed at the bottom of a tube. The gel was layered with 0.5 ml of the bacterial suspension and exposed for 1, 3 or 5 minutes, respectively under slight shaking at room temperature. After exposure 250 μl of the suspension was placed in a new tube, the tube was centrifuged, the supernatant was removed and the cell pellet was resuspended in 250 μl of PBS+10% natural saliva (sterile filtered). Aliquots of 200 μl of the bacterial suspensions were placed into sterile black 96 well plates with clear bottom (Costar® 3603, Corning Inc., USA) and exposed to light from a laser Ceralas G2 (biolitec AG, Germany), 532 nm, power set to 0.05 W, irradiation time of 30 s via an optical fiber from the bottom of the plate. The fluence rate for these settings was about 0.1 W/cm² (measured with Optometer P-9710, Gigahertz-Optik GmbH, Puchheim, Germany). For the used illumination time the resulting total energy fluence was about 3 J/cm².

The control samples for dark toxicity were not exposed to the laser light.

After illumination the samples were removed from the wells of the plate, diluted with Tryptic Soy Broth and plated by using spiral plater Eddy Jet (iul Instruments, Barcelona, Spain) on Tryptic Soy agar plates. The numbers of colony-forming units (CFU/ml) were enumerated after adequate incubation by using colony counter Countermat Flash (iul Instruments, Barcelona, Spain).

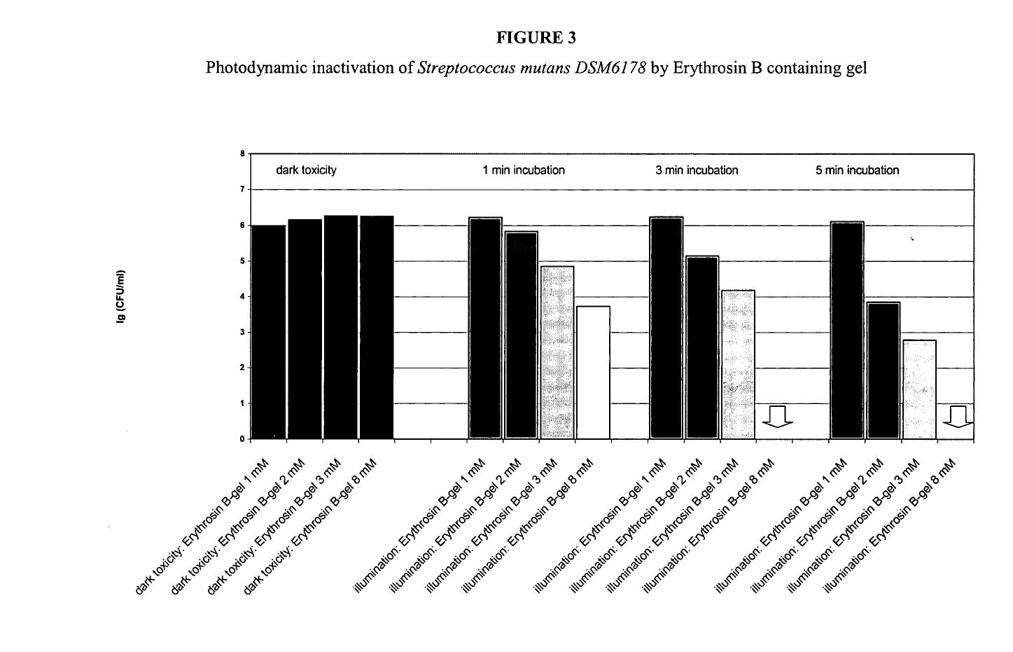

The results of the experiments are shown in FIG. 3:

A very good killing effect by PDT treatment with Erythrosin B containing gel was observed. The antibacterial effect was dependent on the exposure time and on the concentration of Erythrosin B. No dark toxicity was observed.

### EXAMPLE 2

Photodynamic reduction of Streptococcus spec. in the mouth cavity of volunteers 25 volunteers were subdivided in 5 groups. All volunteers applied about 2 ml of Erythrosin B containing gel onto the teeth by gently massaging. After an exposure time of 2 min the mouth cavity was rinsed with water and the teeth were illuminated by light from a 532 nm laser Ceralas G2 (biolitec AG, Germany) by a light applicator via an optical fiber. The irradiation time was about 3 min. The fluence rate of the illumination for the four treated volunteer groups was about 0.05, 0.1, 0.3 and 0.5 W/cm², respectively. The control group of the volunteers was not illuminated. All treatments were done before normal teeth brushing in the morning in order to avoid removing bacteria from the mouth cavity. Before the first treatment, and after every treatment, samples of saliva were taken by Salivette® tubes (Sarstedt Ag & Co., Numbrecht, Germany), the saliva was removed out of the Salivette® tubes and plated by using spiral plater Eddy Jet (iul Instruments, Barcelona, Spain) on TYCSB agar (selective medium for Streptococcus spec.) plates. The numbers of colony-forming units (CFU/ml) were enumerated after adequate incubation in an anaerobic workstation (Don Whithley Scientific Lim., Shipley, England) by using colony counter Countermat Flash (iul Instruments, Barcelona, Spain). For Streptococcus spec. the number of bacteria in the saliva corresponds with the number of bacteria in the plaque of the teeth.

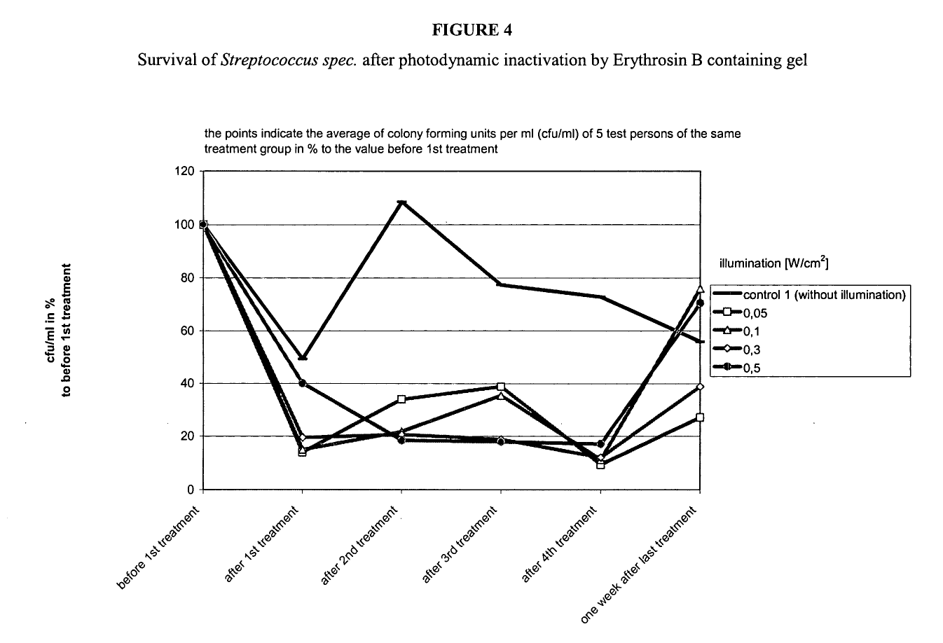

The results of the experiments are shown in FIG. 4:

The best killing effect during the period of treatment was obtained by the illumination with a fluence rate of 0.3 and 0.5 W/cm². A reduction in comparison to the control group was also seen in the group of illumination with 0.1 and 0.05 W/cm².

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of destroying bacteria in a treatment area of a patient comprising the steps of:
a. introducing a composition comprising Erythrosin B as a photosensitizer in a gel to a treatment area on a biological surface;
b. allowing a predetermined time period to elapse to allow said Erythrosin B to couple with bacteria in said treatment area;
c. applying radiation of a preselected wavelength to said treatment area to activate said Erythrosin B and thus stimulating a photodynamic reaction to destroy said bacteria; and
wherein complex media is present in said treatment area.

2. The method of destroying bacteria according to claim 1, wherein said complex media is saliva.

3. The method of destroying bacteria according to claim 1, wherein said treatment area is bacterial plaque on areas selected from the group consisting of teeth and tongue dorsa.

4. The method of destroying bacteria according to claim 1, wherein said treatment area is dental caries.

5. The method of destroying bacteria according to claim 1, wherein said preselected wavelength is between about 500 nm and about 580 nm.

6. The method of destroying bacteria according to claim 1, wherein said preselected wavelength is about 530 nm.

7. The method of destroying bacteria according to claim 1, wherein a concentration of said Erythrosin B in said composition is greater than 1 mM per 0.5 ml of said composition.

8. The method of destroying bacteria according to claim 1, wherein a concentration of said Erythrosin B in said composition is 8 mM per 0.5 ml of said composition.

9. The method of destroying bacteria according to claim 1, wherein said predetermined time period is at least 1 minute.

10. The method of destroying bacteria according to claim 9, wherein said predetermined time period is between 3 minutes and 5 minutes.

11. The method of destroying bacteria according to claim 9, wherein said predetermined time period is at least 5 minutes.

12. The method of destroying bacteria according to claim 1, wherein said applying radiation step is accomplished by a non-coherent lamp.

13. The method of destroying bacteria according to claim 1, wherein said radiation application step is accomplished by an optical transmission system coupled to a radiation source.

14. The method of destroying bacteria according to claim 13, wherein said optical transmission system is at least one optical fiber.

15. The method for destroying bacteria according to claim 1, wherein said radiation is selected from the group consisting of non-coherent radiation and coherent laser radiation.

16. The method of destroying bacteria according to claim 1, wherein said radiation is applied at a fluence rate is at least about 0.05 W/cm².

17. The method of destroying bacteria according to claim 16, wherein said fluence rate is between about 0.3 W/cm² and about 0.5 W/cm².

18. The method of destroying bacteria according to claim 16, wherein a duration of said application of said radiation is about 3 minutes.

19. The method for destroying bacteria according to claim 1, wherein said treatment area is selected from the group consisting of teeth and gums.

20. The method for destroying bacteria according to claim 1, wherein said composition further comprises a material selected from the group consisting of hydroxyethyl cellulose and propyleneglycol.

21. An anti-microbial photodynamic therapy composition for treatment of biological surfaces comprising Erythrosin B and a gel containing a component for solubilizing said Erythrosin B.

22. The anti-microbial photodynamic therapy composition according to claim 21, further comprising a material selected from the group consisting of hydroxyethyl cellulose and propyleneglycol.
